(19) United States
(12) Patent Application Publication
(10) Pub. No.: US 2020/0372639 A1
(43) Pub. Date: Nov. 26, 2020

(54) METHOD AND SYSTEM FOR IDENTIFYING SKIN TEXTURE AND SKIN LESION USING ARTIFICIAL INTELLIGENCE CLOUD-BASED PLATFORM

(71) Applicant: DermAI CO., Ltd., Taipei City (TW)

(72) Inventors: Yu-Chuan Li, Taipei City (TW); Yen-Po Chin, Taipei City (TW)

(73) Assignee: DermAI CO., Ltd., Taipei City (TW)

(21) Appl. No.: 16/831,769

(22) Filed: Mar. 26, 2020

(30) Foreign Application Priority Data
May 24, 2019 (TW) 108118008

(51) Int. Cl. G06T 7/00; G06N 3/08; A61B 5/00

(52) U.S. Cl. CPC G06T 7/0012; G06N 3/08; A61B 5/444; G06T 2207/20084; G06T 2207/30088; G06T 2207/30096; G06T 2207/20081

(57) ABSTRACT

A method and a system for identifying skin texture and skin lesion using artificial intelligence cloud-based platform are provided. The system includes an electronic device and a server. The server includes a storage device and a processor. The processor is coupled to the storage device, and accesses and executes multiple modules stored in the storage device. The multiple modules include an information receiving module, for receiving a captured image and multiple user parameters; a feature vector obtaining module, for obtaining a first feature vector of the captured image and calculating a second feature vector of the multiple user parameters; a skin parameter obtaining module, for obtaining an output result associated with skin parameters according to the first feature vector and the second feature vector; and a skin identification module, for determining a skin identification result according to the output result of the skin parameters.

### CROSS-REFERENCE TO RELATED APPLICATION

This application claims the priority benefit of Taiwan application no. 108118008, filed on May 24, 2019. The entirety of the above-mentioned patent application is hereby incorporated by reference herein and made a part of this specification.

### BACKGROUND

### Technical Field

The disclosure relates to a technology for detecting skin texture and skin lesion, and particularly to a method and a system for identifying skin texture and skin lesion using artificial intelligence cloud-based platform.

### Description of Related Art

In general, in addition to judging the skin condition from the appearance, a dermatologist also comprehensively judges whether the skin has an abnormal condition by consultation. By the appearance and the consultation result, the dermatologist may make a preliminary judgment on the condition of the skin. For example, if a mole on the skin has become significantly larger or has abnormal protrusion over a period of time, there may be a precursor to lesion. Once lesion occurs, it is required to spend time on treatment, causing burden on the body, so early detection of the condition and timely treatment is the best way to avoid suffering.

However, all skin change conditions require the professional judgment of the dermatologist currently. Also, it is normally easy for the user to ignore any skin change and it is difficult to make a preliminary judgement by oneself on whether an abnormal condition of the skin has occurred. Therefore, how to effectively and clearly know the skin condition is one of the problems that persons skilled in the art intend to solve.

### SUMMARY

In view of the above, the disclosure provides a method and a system for identifying skin texture and skin lesion using artificial intelligence cloud-based platform, which can simultaneously consider a skin image and the content of the user's answers to questions to determine a skin identification result by the skin image and user parameters.

The disclosure provides a system for identifying skin texture and skin lesion using artificial intelligence cloud-based platform, which includes an electronic device and a server. The electronic device obtains a captured image and multiple user parameters. The server is connected to the electronic device. The server includes a storage device and a processor. The storage device stores multiple modules. The processor is coupled to the storage device, and accesses and executes the multiple modules stored in the storage device. The multiple modules include an information receiving module, a feature vector obtaining module, a skin parameter obtaining module, and a skin identification module. The information receiving module receives the captured image and the multiple user parameters. The feature vector obtaining module obtains a first feature vector of the captured image and calculates a second feature vector of the multiple user parameters. The skin parameter obtaining module obtains an output result associated with skin parameters according to the first feature vector and the second feature vector. The skin identification module determines a skin identification result corresponding to the captured image according to the output result.

In an embodiment of the disclosure, the operation of the feature vector obtaining module obtaining the first feature vector of the captured image includes: using a machine learning model to obtain the first feature vector of the captured image.

In an embodiment of the disclosure, the operation of the feature vector obtaining module calculating the second feature vector of the multiple user parameters includes: using a vector to represent each of the multiple user parameters; and combining each of multiple vectorized user parameters and inputting each of the multiple vectorized user parameters into a fully connected layer of a machine learning model to obtain the second feature vector.

In an embodiment of the disclosure, the multiple user parameters include a combination of a gender parameter, an age parameter, an affected area size, a time parameter, or an affected area change parameter.

In an embodiment of the disclosure, the operation of the skin parameter obtaining module obtaining the output result associated with the skin parameters according to the first feature vector and the second feature vector includes: combining the first feature vector and the second feature vector to obtain a combined vector; and inputting the combined vector to the fully connected layer of the machine learning model to obtain the output result, wherein the output result is associated with a loss/cost probability of the skin parameters.

In an embodiment of the disclosure, the operation of the skin identification module determining the skin identification result corresponding to the captured image according to the skin parameters includes: determining the skin identification result corresponding to the captured image according to the output result.

In an embodiment of the disclosure, the machine learning model includes a convolutional neural network or a deep neural network.

The disclosure provides a method for identifying skin texture and skin lesion using artificial intelligence cloud-based platform, which is applicable to a server having a processor. The method includes the following steps. A captured image and multiple user parameters are received. A first feature vector of the captured image is obtained and a second feature vector of the multiple user parameters is calculated. An output result associated with skin parameters is obtained according to the first feature vector and the second feature vector. A skin identification result corresponding to the captured image is determined according to the output result.

To make the aforementioned more comprehensible, several embodiments accompanied with drawings are described in detail as follows.

### BRIEF DESCRIPTION OF THE DRAWINGS

FIG. 1 is a schematic diagram of a system for identifying skin texture and skin lesion using artificial intelligence cloud-based platform according to an embodiment of the disclosure.

FIG. 2 is a block diagram of elements of an electronic device and a server according to an embodiment of the disclosure.

FIG. 3 is a flowchart of a method for identifying skin texture and skin lesions using artificial intelligence cloud-based platform according to an embodiment of the disclosure.

FIG. 4 is a flowchart of a method for identifying skin texture and skin lesions using artificial intelligence cloud-based platform according to an embodiment of the disclosure.

### DESCRIPTION OF THE EMBODIMENTS

The disclosure simultaneously considers a skin image and the content of the user's answers to questions to obtain a feature vector of the skin image using a machine learning model and to calculate a feature vector of user parameters. Next, an output result associated with skin parameters is obtained according to the feature vector of the skin image and the feature vector of the user parameters to determine a skin identification result. In this way, the skin image and the content of the user's answers to questions can be simultaneously considered to determine the identification result of skin lesion or skin texture.

Some embodiments of the disclosure will be described in detail with reference to the accompanying drawings. For reference numerals cited in the following descriptions, the same reference numerals appearing in different drawings are regarded as the same or similar elements. The embodiments are only a part of the disclosure and do not disclose all possible implementations of the disclosure. More precisely, the embodiments are merely examples of the method and the system for identifying skin texture and skin lesion using artificial intelligence cloud-based platform in the scope of the present application.

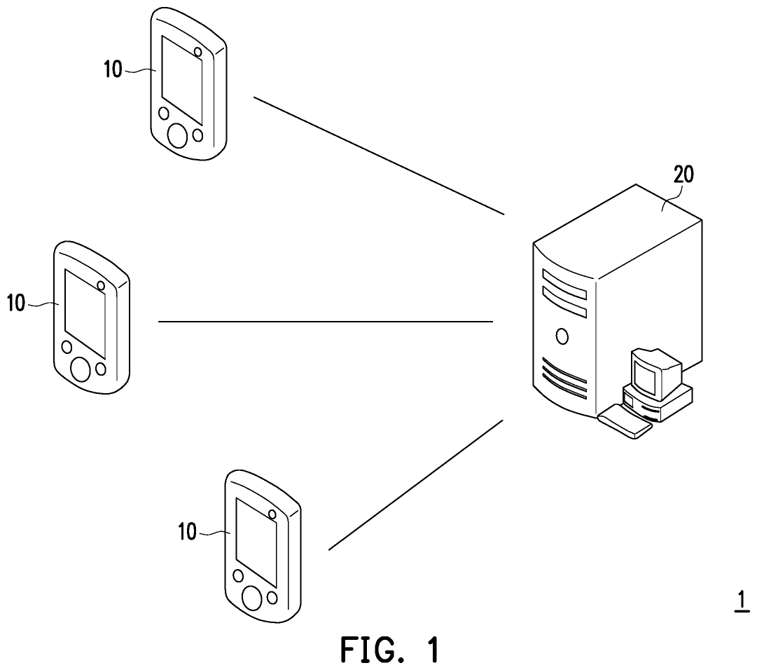

FIG. 1 is a schematic diagram of a system for identifying skin texture and skin lesion using artificial intelligence cloud-based platform according to an embodiment of the disclosure. Referring to FIG. 1, a system for identifying skin texture and skin lesion using artificial intelligence cloud-based platform 1 includes, but is not limited to, an electronic device 10 and a server 20, wherein the server 20 may be respectively connected to multiple electronic devices 10.

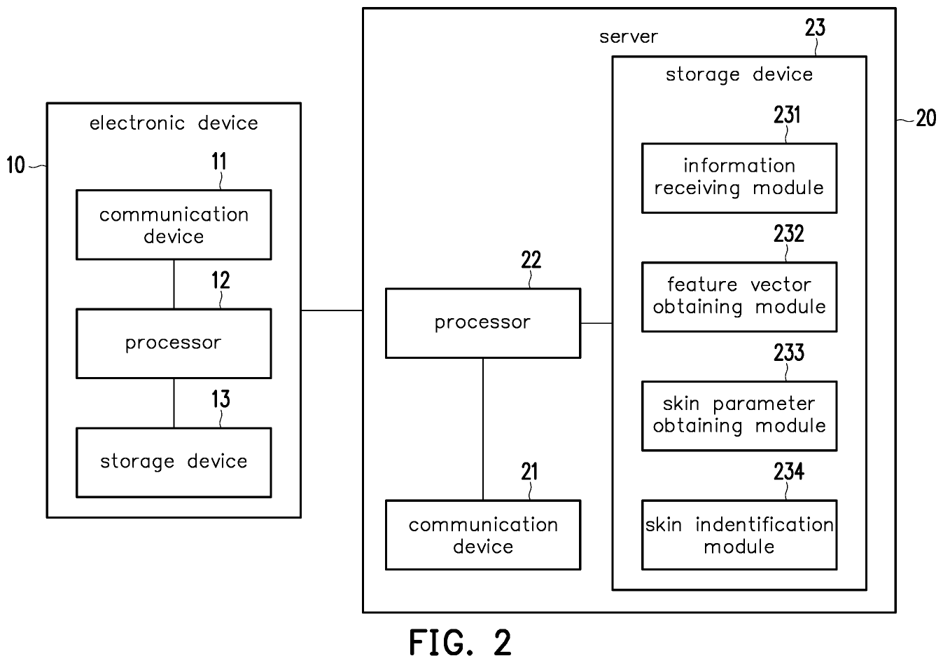

FIG. 2 is a block diagram of elements of an electronic device and a server according to an embodiment of the disclosure. Referring to FIG. 2, the electronic device 10 may include, but is not limited to, a communication device 11, a processor 12, and a storage device 13. The electronic device 10 is, for example, a smart phone, a tablet computer, a notebook computer, a personal computer, or other devices having computing function, but the disclosure is not limited thereto. The server 20 may include, but is not limited to, a communication device 21, a processor 22, and a storage device 23. The server 20 is, for example, a computer host, a remote server, a background host, or other devices, but the disclosure is not limited thereto.

The communication device 11 and the communication device 21 may support communication transceivers such as 3G, 4G, 5G, or later generation mobile communication, Wi-Fi, ethernet, fiber optic network, etc. to connect to the internet. The server 20 communicates with the communication device 11 of the electronic device 10 through the communication device 21 to transmit data to and from the electronic device 10.

The processor 12 is coupled to the communication device 11 and the storage device 13. The processor 22 is coupled to the communication device 21 and the storage device 23. The processor 12 and the processor 22 may respectively access and execute multiple modules stored in the storage device 13 and the storage device 23. In different embodiments, the processor 12 and the processor 22 may respectively be, for example, a central processing unit (CPU), other programmable general-purpose or special-purpose microprocessor, digital signal processor (DSP), programmable controller, application specific integrated circuits (ASIC), programmable logic device (PLD), other similar devices, or a combination of the devices, but the disclosure is not limited thereto.

The storage device 13 and the storage device 23 are, for example, any type of fixed or removable random-access memory (RAM), read-only memory (ROM), flash memory, hard disk, similar elements, or a combination of the elements, and are configured to store programs respectively executable by the processor 12 and the processor 22. In the embodiment, the storage device 23 is configured to store buffered or permanent data, software modules (for example, an information receiving module 231, a feature vector obtaining module 232, a skin parameter obtaining module 233, a skin identification module 234, etc.), and other data or files, and the details thereof will be explained in the following embodiment.

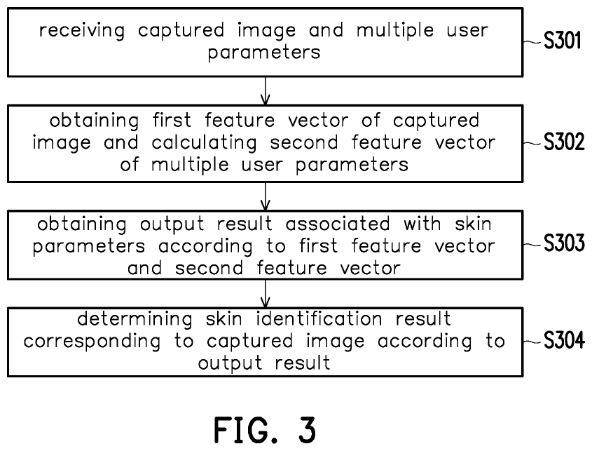

FIG. 3 is a flowchart of a method for identifying skin texture and skin lesions using artificial intelligence cloud-based platform according to an embodiment of the disclosure. Referring to FIG. 2 and FIG. 3 simultaneously, the method of the embodiment is applicable to the system for identifying skin texture and skin lesion using artificial intelligence cloud-based platform 1. The detailed steps of the method for identifying skin texture and skin lesions using artificial intelligence cloud-based platform according to the embodiment will be explained in the following together with various devices and elements of the electronic device 10 and the server 20. Persons skilled in the art should understand that software modules stored in the server 20 do not have to be executed on the server 20, but may also be downloaded and stored in the storage device 13 of the electronic device 10 for the electronic device 10 to execute the software modules to perform the method for identifying skin texture and skin lesion using artificial intelligence cloud-based platform.

First, the processor 22 accesses and executes an information receiving module 231 to receive a captured image and multiple user parameters (Step S301). The captured image and the multiple user parameters may be received by a communication device 21 in a server 20 from a electronic device 10. In an embodiment, the captured image and the multiple user parameters are first obtained by the electronic device 10. In detail, the electronic device 10 is coupled to an image source device (not shown) and obtains the captured image from the image source device. The image source device may be a camera disposed on the electronic device 10, a storage device 13, an external memory card, a remote server, or other devices configured to store an image, but is not limited thereto. In other words, the user, for example, operates the electronic device 10 to capture an image by a camera or operates to obtain a previously captured image from the device, and transmits the selected image to the server 20 as the captured image for use in subsequent operations.

In addition, the server 20 provides multiple questions for the user to answer. After the user answers the questions through the electronic device 10, the result of the answers will be transmitted to the server 20 as user parameters for use in subsequent operations. The user answers the questions through, for example, a user interface displayed by the electronic device 10. The user interface may be a chat room of a communication software, a webpage, a voice assistant, or other software interfaces providing interactive functions, but is not limited thereto.

Then, the processor 22 accesses and executes a feature vector obtaining module 232 to obtain a first feature vector of the captured image and calculates a second feature vector of the multiple user parameters (Step S302).

In detail, in order to obtain the first feature vector of the captured image, the processor 22 first trains parameter values of each layer in a machine learning model through skin lesion image samples and user parameter samples. In an embodiment, the machine learning model is, for example, a machine learning model constructed by a neural network and other technologies. Taking a neural network as an example, many neurons and connections are formed between an input layer and an output layer of the neural network, which may include multiple hidden layers and the number of nodes (neurons) of each layer is uncertain. A larger number of nodes may be used to enhance the robustness of this type of neural network. In the embodiment, the machine learning model is, for example, a convolutional neural network (CNN) or a deep neural network (DNN), but is not limited thereto. Taking the CNN as an example, the parameter values corresponding to skin lesion images may be used as the input of the machine learning model into the CNN. A backward propagation is used for training to use a final loss/cost function to update the parameters of each layer and train the parameter values of each layer in the learning model, wherein a mean square error is regarded as one function. Each skin lesion image sample may be trained using the conventional CNN model structure such as ResNet50, InceptionV3, etc.

The image may then be inputted into the trained machine learning model to obtain an image feature. In an embodiment, the feature vector obtaining module 232 obtains a first feature vector of the captured image by the machine learning model. In other words, after training the machine learning model, the processor 22 inputs the captured image into the trained machine learning model and extracts the first feature vector of the captured image.

On the other hand, the feature vector obtaining module 232 may also calculate a second feature vector of the multiple user parameters. The feature vector obtaining module 232 uses, for example, a vector to represent each user parameter. Each of vectorized user parameters is combined and inputted into a fully connected layer of the machine learning model to obtain the second feature vector. The dimensions of each of the vectorized user parameters after combination are related to the number of questions and the options inside the questions.

In detail, the feature vector obtaining module 232 encodes the user parameters received by the server 20 from the electronic device 10 using an indicator function. For example, if the question is the gender of the user, a vector (1, 0, 0) is generated when the user answers his gender as male; a vector (0, 1, 0) is generated when the user answers her gender as female; and a vector (0, 0, 1) is generated when the user has no intention to answer the gender. After encoding all the user parameters, the feature vector obtaining module 232 combines the encoded user parameters to obtain a combined vector, inputs the combined vector into the fully connected layer for hybridization, and outputs a N-dimensional vector. The fully connected layer considers the interaction between each of the user parameters to generate the second feature vector with more vector dimensions than the vector dimensions of each of the original user parameters. For example, inputting a 16-dimensional vector into the fully connected layer may generate a 256-dimensional vector. In an embodiment, the multiple user parameters include one or a combination of a gender parameter, an age parameter, an affected area size, a time parameter, or an affected area change parameter.

Then, the processor 22 accesses and executes a skin parameter obtaining module 233 to obtain an output result associated with skin parameters according to the first feature vector and the second feature vector (Step S303). The skin parameter obtaining module 233 combines the first feature vector and the second feature vector to obtain a combined vector and inputs the combined vector into the fully connected layer of the machine learning model to obtain the output result, wherein the output result is associated with a loss/cost probability of the skin parameters. In an embodiment, since the first feature vector obtained through the machine learning model may be obtained as a two-dimensional structure picture, the first feature vector may be first converted into a one-dimensional space vector before being combined with the second feature vector to generate the combined vector.

In detail, the skin parameter obtaining module 233 combines the first feature vector of the captured image obtained by the feature vector obtaining module 232 and the second feature vector calculated from the multiple user parameters into the combined vector. Then, the skin parameter obtaining module 233 inputs the combined vector into the fully connected layer and generates the output result at an output layer. The number of output result is related to the intended number of classifications of the output result. Assuming that it is intended to divide the output results into two classifications (for example, no skin condition and with skin condition), then there are two output classifications of the skin parameters at the output layer, but the disclosure does not limit the number of output classifications. The final combined vector inputted into the fully connected layer is converted into the probability (between 0 and 1) of each output classification. In the embodiment, the skin parameters are, for example, different classifications such as “mole with lower risk of malignancy/mole with higher risk of malignancy”, “acne/non-acne”, “good skin condition/bad skin condition”, etc. respectively divided from different output classifications such as “mole”, “acne”, “skin condition”, etc., and the output result is associated with the loss/cost probability of each skin parameter in each output classification.

Finally, the processor 22 accesses and executes a skin identification module 234 to determine a skin identification result corresponding to the captured image according to the output result (Step S304). The skin identification module 234 determines the skin identification result corresponding to the captured image according to the output result. In detail, the classification with the highest probability in the output result is the most likely classification.

Based on the above, according to the embodiments of the disclosure, after inputting the image into the machine learning model to obtain the feature vector of the image and using the fully connected layer to calculate the vectors of the user parameters, the two vectors are combined as data inputted into the fully connected layer of the machine learning model and the output result is generated through the fully connected layer. In other words, in addition to considering picture information, the disclosure also considers non-picture information by establishing the machine learning model capable of simultaneously considering the picture information and the non-picture information, so as to more realistically simulate the situation of clinical judgment of skin texture and to improve the model accuracy.

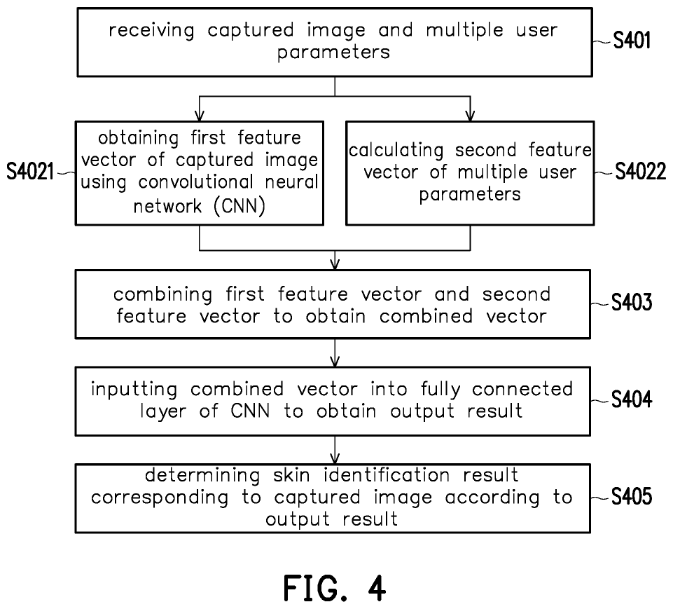

The following embodiment takes “mole” as an example, wherein the output classification “mole” is divided into two skin parameters “mole with lower risk of malignancy” and “mole with higher risk of malignancy”. Also, in the embodiment, the CNN is taken as an example of a machine learning model. FIG. 4 is a flowchart of a method for identifying skin texture and skin lesions using artificial intelligence cloud-based platform according to an embodiment of the disclosure. Referring to FIG. 4, first, a processor 22 receives a captured image and multiple user parameters (Step S401). In the embodiment, the user uses an electronic device 10 to capture or selects the captured image from the electronic device 10. The picture size of the captured image is, for example, set to 224×224 according to a conventional input format and size of the CNN, so the captured image may be represented as a matrix (224, 224, 3), where 3 represents the rank of RGB color. Also, the user answers multiple questions provided by a server 20, wherein the questions include, for example, a combination of “gender (male, female, or no intention to answer)”, “age (under 20 years old, 21-40 years old, 41-65 years old, or above 66 years old)”, “affected area size (0.6 cm or less, or greater than 0.6 cm)”, “period of existence (1 year or less, more than 1 year and less than 2 years, more than 2 years, or did not notice)”, or “affected area change (change in last month, no change in last month, or did not notice)”. The processor 22 receives the captured image and the multiple user parameters transmitted by the electronic device 10.

Then, the processor 22 obtains a first feature vector of the captured image using the CNN (Step S4021). The processor 22 calculates a second feature vector of the multiple user parameters (Step S4022). The processor 22 inputs the captured image into the trained CNN to obtain a first feature vector of the captured image, wherein the CNN is trained using images related to “mole”. After the server 20 receives the user's answers, the processor 22 encodes the answers as vectors. For example, in the embodiment, if the user's answers are male, under 20, 0.6 cm or less, 1 year or less, change in last month, then the vectorized answers are gender (1, 0, 0), age (1, 0, 0, 0), affected area size (1, 0), period of existence (1, 0, 0, 0), and affected area change (1, 0, 0). Then, the processor 22 combines each of vectorized user parameters in terms of dimensions to obtain a combined vector. The processor 22 inputs the combined vector into a fully connected layer of the machine learning model to obtain the second feature vector.

Then, the processor 22 combines the first feature vector and the second feature vector to obtain a combined vector (Step S403). Then, the processor 22 inputs the combined vector into the fully connected layer of the CNN to obtain an output result (Step S404). In the embodiment, the processor 22 combines the first feature vector and the second feature vector in terms of dimensions to obtain the combined vector and inputs the combined vector into the fully connected layer of the CNN to obtain the output result, wherein the output result is associated with a respective loss/cost probability of the two skin parameters “mole with lower risk of malignancy/mole with higher risk of malignancy” in the output classification “mole”.

Finally, the processor 22 determines a skin identification result corresponding to the captured image according to the output result (Step S405). In the embodiment, if the probability of the skin parameter “mole with lower risk of malignancy” in the output result is high, then it is determined that the captured image includes a mole with a lower risk of malignancy. If the probability of the skin parameter “mole with higher risk of malignancy” is high, then it is determined that the captured image includes a mole with a higher risk of malignancy.

In another embodiment, if the CNN is trained using other images related to lesion such as “acne” or images related to skin texture such as “skin condition”, and different questions targeting “acne”, “skin condition”, or other lesion or skin texture are provided as the user parameters for judging lesion or skin texture, then the model established by the system and the method of the disclosure may be configured to assist in judging whether an image of “acne”, “skin condition”, or other lesion or skin texture is compliant with the condition of the specific lesion or skin texture.

In another embodiment, the model for identifying skin texture and skin lesion using artificial intelligence cloud-based platform established by the method for identifying skin texture and skin lesion using artificial intelligence cloud-based platform according to the embodiments of the disclosure may be trained using a backward propagation to use a final loss/cost function to update parameters of each layer, so as to improve the identification accuracy of the model.

Based on the above, the method and the system for identifying skin texture and skin lesion using artificial intelligence cloud-based platform provided by the disclosure can simultaneously consider the skin image and the content of the user's answers to questions, and then input the image into the machine learning model to obtain the feature vector of the image. After the vectors of the user parameters are calculated by the fully connected layer, the feature vector of the image and the vectors of the user parameters are combined as data inputted into the fully connected layer of the machine learning model, and the output result is generated through the fully connected layer. In this way, the probability of each skin parameter can be obtained according to the feature vector of the skin image and the feature vector of the user parameters to determine the identification result of lesion or skin texture. In other words, in addition to considering the picture information, the disclosure also considers the non-picture information by establishing the machine learning model capable of simultaneously considering the picture information and the non-picture information, so as to more realistically simulate the situation of clinical judgment of lesion or skin texture using the condition of affected area and the result of Q&A to improve the model accuracy.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A system for identifying skin texture and skin lesion using artificial intelligence cloud-based platform, comprising:
an electronic device, for obtaining a captured image and a plurality of user parameters; and
a server, connected to the electronic device, the server comprising:
a storage device, for storing a plurality of modules; and
a processor, coupled to the storage device, for accessing and executing the plurality of modules stored in the storage device, the plurality of modules comprising:
an information receiving module, for receiving the captured image and the plurality of user parameters;
a feature vector obtaining module, for obtaining a first feature vector of the captured image and for calculating a second feature vector of the plurality of user parameters;
a skin parameter obtaining module, for obtaining an output result associated with skin parameters according to the first feature vector and the second feature vector; and
a skin identification module, for determining a skin identification result corresponding to the captured image according to the output result.

2. The system for identifying skin texture and skin lesion using artificial intelligence cloud-based platform according to claim 1, wherein the operation of the feature vector obtaining module obtaining the first feature vector of the captured image comprises:
obtaining the first feature vector of the captured image using a machine learning model.

3. The system for identifying skin texture and skin lesion using artificial intelligence cloud-based platform according to claim 1, wherein the operation of the feature vector obtaining module calculating the second feature vector of the plurality of user parameters comprises:
representing each of the plurality of user parameters using a vector; and
combining each of a plurality of vectorized user parameters and inputting each of the plurality of vectorized user parameters to a fully connected layer of a machine learning model to obtain the second feature vector.

4. The system for identifying skin texture and skin lesion using artificial intelligence cloud-based platform according to claim 3, wherein the plurality of user parameters comprise a combination of a gender parameter, an age parameter, an affected area size, a time parameter, or an affected area change parameter.

5. The system for identifying skin texture and skin lesion using artificial intelligence cloud-based platform according to claim 1, wherein the operation of the skin parameter obtaining module obtaining the output result associated with the skin parameters according to the first feature vector and the second feature vector comprises:
combining the first feature vector and the second feature vector to obtain a combined vector; and
inputting the combined vector into a fully connected layer of a machine learning model to obtain the output result, wherein the output result is associated with a loss/cost probability of the skin parameter.

6. The system for identifying skin texture and skin lesion using artificial intelligence cloud-based platform according to claim 5, wherein the operation of the skin identification module determining the skin identification result corresponding to the captured image according to the skin parameters comprises:
determining the skin identification result corresponding to the captured image according to the output result.

7. The system for identifying skin texture and skin lesion using artificial intelligence cloud-based platform according to claim 2, wherein the machine learning model comprises a convolutional neural network or a deep neural network.

8. A method for identifying skin texture and skin lesion using artificial intelligence cloud-based platform, applicable to a server having a processor, the method comprising:
receiving a captured image and a plurality of user parameters;
obtaining a first feature vector of the captured image and calculating a second feature vector of the plurality of user parameters;
obtaining an output result associated with skin parameters according to the first feature vector and the second feature vector; and
determining a skin identification result corresponding to the captured image according to the output result.

9. The method for identifying skin texture and skin lesion using artificial intelligence cloud-based platform according to claim 8, wherein the step of obtaining the first feature vector of the captured image comprises:
obtaining the first feature vector of the captured image using a machine learning model.

10. The method for identifying skin texture and skin lesion using artificial intelligence cloud-based platform according to claim 8, wherein the step of calculating the second feature vector of the plurality of user parameters comprises:
representing each of the plurality of user parameters using a vector; and
combining each of a plurality of vectorized user parameters and inputting each of the plurality of vectorized user parameters into a fully connected layer of a machine learning model to obtain the second feature vector.

11. The method for identifying skin texture and skin lesion using artificial intelligence cloud-based platform according to claim 10, wherein the plurality of user parameters comprises a combination of a gender parameter, an age parameter, an affected area size, a time parameter, or an affected area change parameter.

12. The method for identifying skin texture and skin lesion using artificial intelligence cloud-based platform according to claim 8, wherein the step of obtaining the output result associated with the skin parameters according to the first feature vector and the second feature vector comprises:
combing the first feature vector and the second feature vector to obtain a combined vector; and
inputting the combined vector into a fully connected layer of a machine learning model to obtain the output result, wherein the output result is associated with a loss/cost probability of the skin parameter.

13. The method for identifying skin texture and skin lesion using artificial intelligence cloud-based platform according to claim 12, wherein the step of determining the skin identification result corresponding to the captured image according to the skin parameters comprises:
determining the skin identification result corresponding to the captured image according to the output result.

14. The method for identifying skin texture and skin lesion using artificial intelligence cloud-based platform according to claim 9, wherein the machine learning model comprises a convolutional neural network or a deep neural network.
